(19) United States
(12) Patent Application Publication
(10) Pub. No.: US 2003/0064359 A1
(43) Pub. Date: Apr. 3, 2003

(54) Preparation of highly-purified plasma membranes

(76) Inventors: Joel Lanoix, Montreal (CA); Sylvain Brunet, St - Lambert (CA)

Correspondence Address: CLARK & ELBING LLP, 101 FEDERAL STREET, BOSTON, MA, 02110, US

(21) Appl. No.: 10/251,379

(22) Filed: Sep. 20, 2002

Related U.S. Application Data
(60) Provisional application No. 60/323,849, filed on Sep. 21, 2001.

(51) Int. Cl. C12Q 1/00

(52) U.S. Cl. 435/4

(57) ABSTRACT

The invention features a method for purifying a cell surface plasma membrane, the method including the steps of: (a) contacting a biological sample with an antibody or antigen-binding fragment thereof that specifically binds to a plasma membrane antigen, thereby forming an antibody/plasma membrane complex; and (b) recovering the antibody/plasma membrane complex, thereby purifying the plasma membrane from the biological sample. The invention further features methods for identifying plasma membrane polypeptides using mass spectrometry and purified plasma membrane fractions.

### CROSS REFERENCES TO RELATED APPLICATIONS

This application claims the benefit of U.S. provisional patent application No. 60/323,849 filed on Sep. 21, 2001.

### FIELD OF THE INVENTION

The invention relates to preparation of highly-purified plasma membrane and uses thereof, including mass spectral analysis and detection of differences in plasma membrane content under varying conditions.

### BACKGROUND OF THE INVENTION

Plasma membranes (PM) have previously been purified from various sources including rat liver (Scott et al. (1993) Methods Mol. Biol. 19: 59-69; Hubbard et al. (1983) J. Cell. Biol. 96: 217-229 (PMID: 6298249); Hubbard et al. (1983) J. Cell. Biol. 96: 230-239 (PMID: 6681819)), cultured human cells (Bradley et al. (1994) Methods Enzymol. 228: 432-48 (PMID: 8047013)) or cultured animal cells (Cezanne et al. (1992) Biochem. Biophys. Acta 1112: 205-214 (PMID: 1457453)), as well as from normal colonic cells (Jackson et al. (1977) Cancer 40: 2487-2496 (PMID: 144556); Gustin and Goodman (1981) J. Biol. Chem. 256: 10651-10656 (PMID: 6116709); Brasitus et al. (1983) Biochem. Biophys. Acta 728: 11-19 (PMID: 6830771); Abrahamse et al. (1996) Analytical Biochemistry 242: 112-122 (PMID: 8923973)) and malignant colonic cells (Jackson et al. (1977) Cancer 40: 2487-2496 (PMID: 144556)). To generate a fraction enriched in PMs, these isolation protocols typically use a two-step fractionation involving differential centrifugation coupled to isopycnic centrifugation. These density-based methods are often limited by contaminants in the PM fraction because of the overlapping densities among different organelles.

A variation on this method for purifying PMs involves modifying PM density to allow better separation of the modified PM from contaminating, non-modified organelles. In one approach using differential and isopycnic centrifugations, membrane fractions were incubated in the presence of digitonin to specifically modify the density of the PM and separate it from contaminating organelles (Amar-Costesec et al. (1974) J. Cell Biol. 62: 717-745 (PMID: 4368410)). Digitonin acts as a mild detergent that binds to cholesterol molecules, which are highly abundant at the PM, but depleted from the membranes of other organelles. Treatment with digitonin decreases the intrinsic PM density such that in a second round of isopycnic centrifugation the PM is more cleanly separated from contaminating structures. This approach, however, requires an initial modification of the PM followed by centrifugation, lengthening the procedure. In addition, despite the improvement in purity as compared to centrifugation alone, the PM purity obtained by this method still remains limited.

An alternative to this approach utilizes the properties of gold coupled-wheat germ agglutinin (WGA). In this approach, the density of the PM was specifically increased (Pasquali et al. (1999) J Chromatogr. B Biomed. Sci. Appl. 722: 89-102 (PMID: 10068135)), similarly allowing for better PM separation from other organelle membranes of similar density than centrifugation of unmodified PM. In a similar approach, cationic silica beads have also been used to modify the density of the PM. The positively charged silica beads are used to coat the negatively charged cell surfaces. The residual charged beads are quenched with poly-acrylic polymers prior to homogenization. With this method, PMs are subsequently isolated by low speed centrifugation followed by centrifugation through a nycodenz cushion to allow separation from nuclear contaminants (Chaney and Jacobson (1983) J. Biol. Chem. 258: 10062-10072 (PMID: 6309765)). All of these methods ultimately rely on endogenous or experimentally-induced density differences between PM and various organelles to permit separation by centrifugation with varying degrees of contamination. Each of these methods also lacks the ability to distinguish and separate the PMs of specific cell types, as would be necessary for experiments involving the isolation of PM from tissue composed of several distinct cell types.

Immunoisolation is a different approach that has been used to purify various intracellular membrane compartments (Luers et al. (1998) Electrophoresis 19: 1205-1210 (PMID: 9662184); Calhoun and Goldenring (1997) Biochem J. 325: 559-564 (PMID: 9230141); Jin et al. (1996) J. Biol. Chem. 47: 30105-30113 (PMID: 8939959); Saucan and Palade (1994) J. Cell Biol. 125: 733-741 (PMID: 8188743); Henley et al. (1996) J. Cell. Biol. 133: 761-775 (PMID: 8666662)). This method relies on the use of high affinity antibodies to specifically recognize antigens intrinsic to the organelle to be isolated. Through high-affinity interactions with the antigen, the antibody functions as a molecular hook, allowing the organelle to be specifically isolated from other cellular material. Retrieval and isolation of the antibody-antigen immuno-complex from a crude cell extract is generally performed using a secondary antibody (an antibody that specifically binds to the first (primary) antibody) coupled to a solid support. In some cases the solid support then provides a density basis for separation by centrifugation. Immunoisolation has been previously utilized for the isolation of PMs, however, the methods used resulted in contamination of the PM fraction by various other organelles (Devaney and Howell (1985) EMBO J 4: 3123-3130 (PMID: 4092679); Gruenberg and Howell (1985) Eur J. Cell Biol. 38: 313-321 (PMID: 2995034); Gruenberg and Howell (1986) EMBO J 5: 3901-3101 (PMID: 3028771)).

Although moderately enriched PM fractions are obtained using these immunoisolation approaches, none afford the isolation of either an intact PM or an intact PM from a specific type of cell or tissue without prior cell sorting. As a result, these methods have been generally unsatisfactory lacking the efficiency and sensitivity to rapidly separate and purify PMs from cultured cells or primary tissue or both. Furthermore, the immunoisolation methods described above do not afford sufficient purity for mass spectral analyses and subsequent protein identification.

With the advent of new protein identification technologies such as mass spectrometry, it is desirable to obtain highly purified organelle fractions from cells important in health and disease in order to comprehensively identify all of the proteins present. The complete isolation of specific organelles with minimal contamination becomes critically important for the assessment of the absence or presence and relative levels of a protein. These assessments are used to make comparisons between proteins in health, disease, and under treatment conditions and may also help to identify specific proteins as important diagnostic markers, prognostics of disease, or as therapeutic targets.

Furthermore, the plasma membrane is an ideal location for the identification of potential therapeutic targets. The identification of such targets on the cell surface is important because it could obviate the need for the delays and difficulties that can accompany designing cell entry technologies for drugs. For example, important drug targets, such as G-protein coupled receptors or growth factor receptors, reside at the cell surface embedded within the PM. Also, for most antibody-based therapies, it is thought that the most effective antigen targets reside on the surface of cells, i.e., within the PM and facing the extracellular space.

For these and other reasons, there is universal interest in identifying the complete set of protein components that comprise the PM of important cells and tissues of the body in health and disease. In addition, in order to be amenable to study by mass spectrometry, it is preferable that the purified PM preparations be isolated at a sufficient scale, specific to a unique cell type, and as devoid of other contaminating organelles as possible. Thus, there is a need in the art to improve PM purification methodologies.

### SUMMARY OF THE INVENTION

The applicant has developed a systematic method for purifying PMs from a variety of biological samples, including cell cultures and primary tissues such as colon tumors. In one aspect, the invention features a method for purifying a cell surface PM. The method, in general, includes the steps of: (a) contacting a biological sample with an antibody or antigen-binding fragment thereof that specifically binds to a PM antigen, thereby forming an antibody/PM complex; and (b) recovering the antibody/PM complex, thereby purifying the PM from the biological sample. In preferred embodiments, step (a) further includes incubating the antibody/PM complex, or antigen-binding fragment/PM complex, with an insoluble affinity support reagent (such as paramagnetic beads, paramagnetic microbeads, or sepharose beads) that specifically binds antibody or antigen-binding fragments thereof. Preferably, the antibody or antibodies used in the methods are monoclonal or polyclonal antibodies and are directed to a subdomain antigen of the PM (for example, the apical or basolateral subdomains). Exemplary antibodies useful in the methods include monoclonal anti-ESA and anti-CEA antibodies (see abbreviations listed below). In other preferred embodiments, the biological sample is a cell culture (for example, from a normal or diseased cell); a primary tissue; or a population of tumor cells (for example, carcinoma cells such as cells derived from a carcinoma having a primary site of breast, ovary, stomach, intestine, colon, pancreas, or lung tissue). For any of the biological samples, the sample can be prepared as a single cell suspension prior to the contacting of the sample with the antibodies or antigen-binding fragments thereof.

In preferred embodiments the insoluble affinity support is a paramagnetic bead that can range in diameter from about 5 μm or less to about 50 nm or less. The insoluble affinity support can also be a sepharose bead. In additional preferred embodiments, the paramagnetic beads are first coupled to the secondary antibody prior to complexing with the antibody/PM immunocomplex. Alternatively, the paramagnetic beads conjugated to secondary antibody are first complexed to primary antibody prior to addition to PM samples.

In other aspects, the invention features a method for analyzing PM polypeptides by mass spectrometry. The method, in general, includes the steps of purifying PMs according to the above-described methods and analyzing the purified PM polypeptides by mass spectrometry.

In yet other aspects, the invention features a purified PM fraction. Such a purified PM fraction is preferably at least 70% pure, as determined according to the methods described herein. More preferably, the purified PM fraction is at least 75%, 80%, or 85% pure. More preferably still, the fraction is at least 86%, 87%, 88%, 89%, 90%, 91%, 92%, 93%, 94%, or 95% pure. Most preferably, the fraction is of a purity of at least 96%, 97%, 98%, 99%, or better. In preferred embodiments, the purified PM fraction is obtained from PMs of cultured cells, a population of tumor cells, or from cells of a primary tissue. In other preferred embodiments, the purified PM fraction includes PMs having microvilli or a clathrin-coated morphology or both.

The invention also features a purified magnetic bead/PM immunocomplex. Such an immunocomplex can include any or all of the following: the primary antibody, the secondary antibody, the purified PM fraction, and the magnetic beads. In preferred embodiments, the PM is obtained from PMs of cultured cells, a population of tumor cells or from cells of a primary tissue. In other preferred embodiments, the PM fraction includes PMs having microvilli or a clathrin-coated morphology or both. Such a purified magnetic bead/PM immunocomplex is preferably at least 70% pure, as determined according to the methods described herein. More preferably, the immunocomplex is at least 75%, 80%, or 85% pure. More preferably still, the immunocomplex is at least 86%, 87%, 88%, 89%, 90%, 91%, 92%, 93%, 94%, or 95% pure. Most preferably, the immunocomplex is of a purity of at least 96%, 97%, 98%, 99%, or better.

This invention provides a number of advantages and uses over conventional methods. For example, the methods disclosed herein provide for the preparation of highly-purified PMs. In addition, the invention provides for the isolation of an entire, intact PM, as well as for the purification of the PM of specific cell types from a heterogeneous population of cells, and in sufficient quantity and purity to perform mass spectral analysis followed by protein identification. In addition, the methods of the invention are rapid and involve relatively few steps. Furthermore, the methods described herein enable the purification of biochemically useful quantities of intact PMs from relatively small amounts of starting samples. For example, the invention allows for very sensitive detection and purification of PMs from metastatic tumor cells. Selected antibodies bind their cognate antigens at the cell surface with sufficient specificity to, for example, isolate and purify PMs of tumor cells away from PMs of stromal cells. In addition, the plasma membranes are easily separated from contaminating membranous intracellular organelles. Furthermore, the purified membranes allow for the identification of the entire protein complement of the PM. Such information is useful, for example, in the diagnosis and treatment of disease and for the identification of novel therapeutic targets.

Moreover, utilizing purified plasma membrane preparations and mass spectrometry to identify important proteins that are diagnostic or therapeutic targets has many advantages. For example, as compared to a total cell lysate, the use of purified organelles enriches for low abundance proteins. In addition, to the enhanced detection of low abundance proteins, a greater proportion of total PM proteins may be identified. And, the ability to localize unknown or unexpected proteins to an organelle of known function, yields insights into that proteins' function. Finally, the specific location and orientation of proteins within the cell are important considerations for validating a protein as a valuable drug target.

Abbreviations and Definitions

The following abbreviations and definitions are used.

2D CE-IMLS: 2 Dimensional Capillary Electrophoresis with Inverted Mass Ladder Sequencing

ATCC: American Type Culture Collection

CEA: carcinoembryonic antigen

CE-MS: Capillary Electrophoresis Mass Spectrometry

DMEM: Dulbecco's Modified Eagle Medium

DTE: dithioerythritol

EGTA: ethylene glycol-bis[β-aminoethyl ether]-N,N,N′,N′-tetra-acetic acid

ESA: epithelial specific antigen, also known as major gastrointestinal tumor-associated protein GA733-2 precursor, (epithelial cell surface antigen), (epithelial glycoprotein) (EGP), KSA, tumor-associated calcium signal transducer 1 precursor, Ep-CAM/EGP40

ESI: ElectroSpray Ionization Mass Spectrometry

ESI-MS/MS: ElectroSpray tandem Mass Spectrometry

ESI-TOF: ElectroSpray Ionization-Time-of-Flight

FAB/MS: Fast Atom Bombardment Mass Spectrometry

FACS: Fluorescence Activated Cell Sorting

FBS: Fetal Bovine Serum

FCS: Fetal Calf Serum

FIA/MS: Flow Injection Analysis Mass Spectrometry

FT/ICR: Fourier Transform-Ion Cyclotron Resonance Mass Spectrometry

HCl: hydrochloric acid

HEPES: N-[2-hydroxyethyl]piperazine-N′-[2-ethanesulfonic acid]

IgG: an immunoglobulin with class G heavy chains

IgM: an immunoglobulin with class M heavy chains

LC/MS: Liquid Chromatography/Mass Spectrometry

MALDI: Matrix-Assisted Laser Desorption Ionization Mass Spectrometry

MALDI-TOF: Matrix-Assisted Laser Desorption Ionization-Time-of-Flight

MALDI-TOF/PMF: Matrix-Assisted Laser Desorption Ionization-Time-of-Flight/Peptide Mass Fingerprinting

MIMS: Multi-Isotope Imaging Mass Spectrometry

MS: Mass Spectrometry

MS/MS: Tandem Mass Spectrometery

MS/MS/MS: Multiple Mass Spectrometry

PBS: phosphate-buffered saline

PES: polyethersulfone

PIMS: Photoionization Mass Spectrometry

PM: Plasma Membrane

PMID: PubMed Identification Number

PNS: Post-Nuclear Supernatant

PVP: polyvinyl-pyrrolidone

PyMS: Pyrolysis Mass Spectrometry

REMPI: Resonance Enhanced MultiPhoton Ionization

SIFT-MS: Selected Ion Flow Tube Mass Spectrometry

SIMS: Secondary Ion Mass Spectrometry

ST: sucrose-tris

TOF: Time-of-Flight

Tris: 2-amino-2-(hydroxymethyl)-1,3-propanediol

As used herein, “affinity” refers to strength of binding of one compound to another. For example, a compound with a high affinity for a specific protein can be used to readily purify that protein from a mixture. A lower affinity compound might be used, for example, if it gave a desirable broader specificity, such as allowing several members of a particular protein family to be isolated. A high binding affinity is generally desired between an antibody and its antigen. Binding strength can be influenced by experimental conditions and is determined according to standard methods.

As used herein, “affinity reagents” refer to compounds, substances, and apparati, which function, at least in part, through the particular characteristics of their binding affinity, typically for a specific target. Exemplary affinity reagents include, without limitation, antibodies and antigen-binding fragments thereof.

As used herein, “antibody” refers to an immunoglobulin protein whether naturally or synthetically produced, which is capable of binding an antigen. Such an antibody may be a complete antibody, as well as antigen-binding fragments thereof (e.g., F(ab′)₂, Fab,) and modified antibodies produced therefrom (e.g., antibodies modified through chemical, biochemical, or recombinant DNA methodologies), provided that the antigen-binding fragments and modified antibodies retain antigen binding characteristics sufficiently similar to the template antibody so as to provide for specific binding of antigen. Antibodies may be obtained from any source, including, but not limited to, hybridomas, mice, rabbits, humans, monkeys, chickens, goats, rats, and the like. The term antibody also covers any recombinant protein having a binding domain that is based on or derived from sequences of an immunoglobulin binding domain, such as an antigen-binding fragment of the antibody. The term also includes antibodies that have been chemically modified, such as a cyclized peptide.

As used herein, “antigen-binding fragment” refers to a portion of an antibody (e.g., Fv) capable of binding to an antigen. Such a fragment may be generated by proteolysis of an antibody, or recombinantly produced using sequence derived from the antibody protein or a nucleic acid encoding the protein.

As used herein, “antigen” refers to a substance that, upon introduction or injection into a vertebrate animal such as a mammal or poultry, or presentation by antigen presentation machinery, stimulates the animal to produce antibodies that can recognize or bind to the antigen. Such antigens are derived from a variety of sources and include, for example, viruses, proteins, nucleic acids, organic compounds, and the like.

As used herein, “anti-CD-104” refers to an antibody or antigen-binding fragment thereof that binds to the human CD-104 protein (e.g., GenBank Accession number P16144), its precursor, isoforms, fragments thereof, orthologues thereof, or any variation, including, but not limited to, polymorphisms, mutations, and sequence corrections.

As used herein, “anti-CD-138” refers to an antibody or antigen-binding fragment thereof that binds to the human CD-138 protein (e.g. GenBank Accession number P18827), its precursor, isoforms, fragments thereof, orthologues thereof, or any variation, including, but not limited to, polymorphisms, mutations, and sequence corrections.

As used herein, “anti-CD-164” refers to an antibody or antigen-binding fragment thereof that binds to the human CD-164 protein (e.g. GenBank Accession number NP_{—}006007), its precursor, isoforms, fragments thereof, orthologues thereof, or any variation, including, but not limited to, polymorphisms, mutations, and sequence corrections.

As used herein, “anti-CEA” refers to an antibody or antigen-binding fragment thereof that binds to the human CEA protein (e.g., GenBank Accession numbers A36319 and AAA51965), its precursor, isoforms, fragments thereof, orthologues thereof or any variation, including, but not limited to, polymorphisms, mutations, and sequence corrections. For example, CEA Ab-3 monoclonal antibody (Neomarkers, Fremont, Calif.) is an anti-CEA antibody.

As used herein, “anti-ESA” refers to an antibody or antigen-binding fragment thereof that binds to the human ESA protein (GenBank Accesion number P16422), its precursor, isoforms, fragments thereof, orthologues thereof or any variation, including, but not limited to, polymorphisms, mutations, and sequence corrections. For example, ESA Ab-3 monoclonal antibody (NeoMarkers, Fremont, Calif.) is an anti-ESA antibody.

As used herein, the term “binding” refers to a covalent or non-covalent interaction that joins two or more molecules together. An example of two such molecules is an enzyme and an inhibitor of that enzyme. Another example is an enzyme and its substrate. A third example is an antibody and an antigen. Non-covalent interactions include hydrogen bonding, ionic interactions among charged groups or dipoles, van der Waals interactions, and hydrophobic interactions among non-polar groups. One or more of the afore-mentioned interactions can mediate the binding of two or more molecules to each other. Binding also can be influenced by experimental conditions.

As used herein, “biological sample” refers to a whole organism or portion thereof, such as a quantity of body fluid, cells, cell lysate, extracellular matrix, tissue, organ, and/or combinations thereof. Cells from cell culture are also contemplated. Samples may be live or dead, or taken from a living or deceased organism or organisms. Samples may be of normal, abnormal, or diseased tissue. They may also be derived from a number of possible treatment conditions not limited to environmental, chemical, drug treatment, and the like.

As used herein, “cell surface-binding” refers to the ability to bind the PM or a component thereof.

As used herein, “clathrin-coated morphology” refers to the characteristic morphology of the PM where clathrin is localized on the inner surface of the cell membrane, particularly in the form of a “clathrin-coated pit,” an invagination of the PM where clathrin is localized. The polypeptides on the inner surface of the cell membrane typically exhibit a localized and characteristic thickening or coating of the cell membrane that can be visualized by electron microscopy.

As used herein, “epitope” refers to a molecular region on an antigen capable of eliciting an immune response and of binding with the specific antibody produced by the response. In the animal, most antigens will present several epitopes or antigenic determinants simultaneously, depending on the size and immunogenicity of the antigen or antigenic molecule. Regions of protein sequence may be capable of adopting different conformations, of varying binding affinity for a particular antibody, and varying immunogenicity. Under certain circumstances, it may be necessary or advantageous, to conformationally constrain a particular epitope to produce an antibody selective for a particular antigen conformation.

As used herein, the term “immune response” refers to the series of molecular events that are elicited when an antigen is encountered by the immune system. Such molecular events include the expansion of B- and T-cells and the production of antibodies.

As used herein, “insoluble affinity support reagent” refers to an affinity reagent that does not disintegrate into smaller pieces through solvent action, beyond a known configuration in common biological solutions or lysates. Exemplary insoluble affinity support reagents include paramagnetic beads or paramagnetic microbeads, which are not likely to break down in solution over the course of an average experiment.

By “intact plasma membrane” is meant a PM having no relevant component (for example, the apical and basolateral domains) substantially removed or destroyed. By “apical plasma membrane domain” is meant the sub-domain of the PM that is facing the lumen. By “basolateral plasma membrane domain” is meant the sub-domain of the PM that underlies the connective tissue (basal domain) as well as the sub-domain of the PM facing adjacent cells (lateral domain).

As used herein, “mass spectrometry” or “MS” refers to a technique for measuring and analyzing molecules involving introducing enough energy (e.g., ionization) into a target molecule to cause its disintegration. The resulting fragments are then analyzed based on their mass/charge ratios, which can produce a “molecular fingerprint.” The instrument used to perform this analysis is most frequently a mass spectrometer. Generally a mass spectrometer couples three devices: an ionization device, a mass analyzer, and a detector. The most common ionization techniques used are MALDI and ESI. Once a sample has been ionized, its mass is then analyzed. The most commonly used mass analyzers for protein biochemistry applications are TOF, triple-quadrupole, quadrupole-TOF, and ion trap instruments. Herein, MS refers to the basic techniques and improvements and variations thereupon commonly known in the art, including, but not limited to, 2D CE-IMLS, Affinity Mass Spectrometry, CE-MS, ESI, ESI-MS/MS, ESI-TOF, FAB/MS, FIA/MS, FT-ICR, Hybrid Mass Spectrometry, Ion Trap Mass Spectrometry, Liquid Chromatography/Mass Spectrometry (LC/MS), MALDI, MALDI-TOF, MALDI-TOF/PMF, MIMS, MS/MS, MS/MS/MS, NanoElectroSpray-MS/MS, PIMS, PyMS, Quadrupole Ion Trap, REMPI, SIFT-MS, SIMS, TOF, and triple quadrupole.

As used herein, “microvilli” refers to microscopic projections of a cell, especially any of the fingerlike outward projections of some epithelial cell surfaces. Microvilli are covered with cell membrane and their cytoplasm is continuous with the main cell cytoplasm. The presence of microvilli may be assessed by any methods known in the art, though most commonly their presence is noted by cell biological methods such as electron microscopy.

As used herein, “monoclonal” refers to an antibody produced from a cell clone and therefore includes a single type of immunoglobulin. Monoclonal antibodies are generally produced by fusing antibody-forming lymphocytes from mouse spleen with mouse myeloma cells. The resulting hybrid cells multiply rapidly and produce the same antibody as their parent lymphocytes.

As used herein, “organelle” refers to a subcellular compartment or structure of eukaryotic cells with a discrete function, usually bound by a membrane. Membranous organelles include, but are not limited to, mitochondria, chloroplasts, peroxisomes, lysosomes, endoplasmic reticulum, phagosomes, endosomes, nuclei, nucleoli, plastids, vesicles, secretory vesicles, vacuoles, and Golgi apparatus. Non-membranous organelles include, but are not limited to, the cytoskeleton, nuclear pores, centrioles, ribosomes, desmosomes, gap junctions, tight junctions, cilia, flagella, and proteasomes.

As used herein, “paramagnetic beads” refers to microscopic affinity reagents that allow binding to an antibody or antibody/antigen complex or another affinity reagent attached thereto and permit the isolation of the resulting complex through the susceptibility to isolation by magnetism of the beads. Beads need not be bead-like, e.g., of spherical shape, but often are. Beads may of about 5.0 μm or less in diameter if spherical or roughly so, or of about 5.0 μm or less along the longest axis for those of non-spherical geometry. An example of paramagnetic beads conjugated to an antibody is Pan anti-mouse IgG Dynabeads (Dynal Biotech, Lake Success, N.Y.).

As used herein, “paramagnetic microbeads” refers to microscopic affinity reagents that allow binding to an antibody or antibody/antigen complex or another affinity reagent attached thereto and permit the isolation of the resulting complex through the susceptibility to isolation by magnetism of the beads. Microbeads need not be bead-like, e.g., of spherical shape, but often are. Microbeads may of about 100 nm or less in diameter if spherical or roughly so, or of about 100 nm or less along the longest axis for those of non-spherical geometry. An example of paramagnetic microbeads conjugated to an antibody is MACS Goat anti-mouse IgG Microbeads (Miltenyi Biotec, Gladbach, Germany).

As used herein, “plasma membrane”, also called “PM,” “cell membrane,” or “plasmalemma,” refers to a semi-permeable boundary layer of a cell. The current model of the plasma membrane is of a fluid mosaic composed of a bilayer of phospholipids (with cholesterol in some types) oriented with their hydrophilic heads toward the membrane's surface and their hydrophobic tails towards the membrane's interior, together with a variety of proteins. Some animal cells may have an additional outer layer of carbohydrates, termed a glycocalyx. Plasma membranes are dynamic structures, serving not only as an envelope for the cell but also as a selective barrier for regulating the passage of substances into and out of the cell.

As used herein, “plasma membrane antigen” refers to an antigen present in all or part of a plasma membrane, an attachment thereto, or a component thereof, such as an integral membrane protein.

As used herein, “plasma membrane fraction” refers to a fraction of cellular material or cell lysate containing immunoisolated plasma membrane.

As used herein, “plasma membrane polypeptides” refers to polypeptides present in a plasma membrane fraction.

As used herein, “polyclonal” generally refers to an antibody produced through an elicited immune response, consisting of one or, likely, more types of immunoglobulin. The immune response may be elicited in any animal, including, but not limited to, goats, rats, rabbits, mice, and chickens. Such antibodies are usually present in sera, but may be purified (including partially) for example, through a process selectively enriching for immunoglobulins. They may also be affinity purified (including partially) through a process selectively enriching for binding to an antigen.

By “primary tissue” is meant any tissue obtained directly from an animal such as a human patient. Such tissue includes epithelial tissue, including covering, lining and glandular epithelial tissue; connective tissue, including fluid, supportive and adipose tissue; muscle tissue, including skeletal, cardiac and smooth muscle; and nervous tissue.

As used herein, “protein” or “polypeptide” or “peptide” refers to any of numerous naturally occurring, sometimes extremely complex (such as an enzyme or antibody) substances that consist of a chain of four or more amino acid residues joined by peptide bonds. The chain may be linear, branched, circular, or combinations thereof. Intra-protein bonds also include disulfide bonds. Protein molecules contain the elements carbon, hydrogen, nitrogen, oxygen, usually sulfur, and occasionally other elements (such as phosphorus or iron). Herein, “protein” is also considered to encompass fragments, variants and modifications (including, but not limited to, glycosylated, acylated, myristylated, and/or phosphorylated residues) thereof, including the use of amino acid analogs, as well as non-proteinacious compounds intrinsic to enzymatic function, such as co-factors, or guide templates (for example, the template RNA associated with proper telomerase function). “Peptide” is often used more commonly to refer to a protein with a small number of amino acids or whose sequence is a smaller subset of that of a larger protein.

By “purified plasma membrane” is meant a PM fraction that is substantially devoid of other non-contiguous intracellular membranous organelle contaminants such as nuclei, mitochondria, lysosomes, peroxisomes, rough and smooth endoplasmic reticulum, and Golgi apparatus. The purity of such fractions is typically assessed using a standard quantitative morphometric and/or biochemical analysis. Such purified PM fractions are generally at least 85%, more preferably 90%, and most preferably 95% free of the contaminating membranous organelles with which they are naturally associated.

As used herein, “secondary antibody” refers to an antibody or antibodies that can recognize specific types of immunoglobulins. For example, goat anti-mouse IgG is immunoglobulin produced by goats that specifically binds to G-type immunoglobulins produced by mice. It should be noted that a “secondary” antibody should be distinguished from a “second” antibody, which need not have an immunoglobulin as its antigen.

As used herein, “specific binding,” or when something is said to “specifically bind,” “specifically recognize,” and/or “specifically interact,” refers to binding, even briefly, between a compound, such as an antibody, and one or more other substances, molecules, and/or compounds wherein the binding interaction is dependent upon the conformation or structure of the molecules, such as that determined by primary amino acid sequence. Specificity may be to a broad class of compounds, such as is generally the case for secondary antibodies, which may bind all the various IgG molecules present in a particular species. It may also refer to binding to self, or other molecules of the same protein, as in the forming of dimers and other multimers. Similarly, selective binding is based on affinity, with a substance exhibiting a generally higher affinity for a particular molecule than for other molecules commonly present under the appropriate experimental conditions.

Other features and advantages of the invention will be apparent from the following detailed description and from the claims.

### BRIEF DESCRIPTION OF THE DRAWINGS

FIG. 1 shows the biochemical characterization of an immunoisolated PM fraction from CaCo-2 cells by western blotting, using antibodies that specifically recognize PM antigenic markers (ESA, Na⁺/K⁺ ATPase, CEA, and annexin II), as well as markers of intracellular contaminating organelles (BIP, TOM 20, p251, and p62 nucleoporin). Lane 1: total cell lysate; Lane 2: ESA/CEA bead fraction; Lane 3: control IgGs bead fraction.

FIG. 2 shows the morphology of PM purified from CaCo-2 cells. A) Morphological characterization electron micrographs showing microvilli, a marker of the PM. B) Morphological characterization: low and high magnification electron micrographs showing immunoisolated PM bound to magnetic beads coupled with the monoclonal ESA and CEA antibodies. Clathrin-coated pits, markers of PM, are noted with “Cp”.

FIG. 3 shows the biochemical characterization of an immunoisolated PM fraction from primary human colon tumor tissue by western blotting, using antibodies that specifically recognize PM markers (Na⁺/K⁺ ATPase, CEA, and annexin II), as well as markers of intracellular contaminating organelles (p62 nucleoporin, TOM 20, and p251). Lane 1: Total cell lysate, normal tissue; Lane 2: PM fraction, normal tissue; Lane 3: Control IgG fraction, normal tissue; Lane 4: Total cell lysate, tumor tissue; Lane 5: PM fraction, tumor tissue; Lane 6: Control IgG fraction, tumor tissue.

FIG. 4 shows a series of electron micrographs depicting the material bound to magnetic beads following the immunoisolation purification protocol from primary human colon tumor tissue (left, center, and right panels). Also noted are morphological PM markers such as clathrin-coated structures (Ccs, left panel) and microvilli (right panel).

FIG. 5 shows the biochemical characterization of a microbead immunoisolated PM fraction from Caco-2 cells by western blotting, using antibodies that specifically recognize PM markers (Na⁺/K⁺ ATPase and CEA), as well as markers of intracellular contaminating organelles (p62 nucleoporin and BIP). Lane 1: total cell lysate; Lane 2: ESA/CEA bead fraction.

FIG. 6 shows a series of electron micrographs depicting the PM material from Caco-2 cells bound to magnetic microbeads following the immunoisolation purification protocol of Example 5.

FIG. 7 shows the biochemical characterization of an immunoisolated PM fraction from primary human colon tissue by western blotting, using antibodies that specifically recognize PM markers (Na⁺/K⁺ ATPase and CEA), as well as markers of intracellular contaminating organelles (p62 nucleoporin and BIP). Lane 1: Total cell lysate from normal cells. Lane 2: Immunoisolated PM fraction from normal cells. Lane 3: Total cell lysate from tumor cells. Lane 4: Immunoisolated PM fraction from tumor cells.

### DETAILED DESCRIPTION OF THE INVENTION

Methods are provided that utilize affinity reagents (e.g., an antibody or an antigen-binding fragment thereof) to purify PMs from biological samples of interest, for example, PMs of cultured cells or PMs of cells comprising a primary tissue. The methods, in general, involve the binding of at least one antibody, or antigen-binding fragment thereof, to a plasma membrane antigen. PM antigen-binding antibodies may be of the IgG or IgM type or may be a fragment of an IgG or IgM antibody. Preferably the antibody specifically recognizes one or more antigens present on cell surface PMs, and not on the membranes of intracellular membranous organelles such as endoplasmic reticulum, nuclei, Golgi apparatus, endosomes, lysosomes, mitochondria, or peroxisomes. In other embodiments, the antibody recognizes one or more antigens on the membranes of an intracellular membranous organelle.

In preferred embodiments, the immunoisolation of PMs relies on high-specificity antibodies to PM antigens. By “high-specificity antibodies” is meant antibodies that preferentially recognize their cognate antigens and possess limited cross-reactivity with other irrelevant proteins. The most preferable antibody is a monoclonal antibody. Polyclonal antibodies can also be used and it is preferred that such polyclonal antibodies be purified (e.g., affinity purified). High-specificity antibodies preferably bind to antigens specifically localized at the outer surface of the PM and, importantly, to antigens expressed only in the PM of the cell type to be isolated.

The latter parameter is crucial to obtain a fraction free of PMs derived from stromal cells that are always present in variable proportion in a primary tissue. For example, the epithelial cells in a normal colon tissue represent approximately 10% of the total population of cells. The remainder of the cellular population consists of muscle cells, fibroblasts, and endothelial cells. In order to isolate the PM of a specific cell type, monoclonal antibodies against specific PM antigens present exclusively on the PM of the cell of interest are used. Accordingly, any selected combination of antibodies recognizing a proper set of antigens could be used for immunoisolating the PMs of any cultured cell type and/or primary tissue (normal or diseased) that might be found in a biological sample. For example, PMs may be purified from prostate primary tissue or prostate cells (such as LNCap cells) using the monoclonal antibody 3C6 which recognizes the Prostate-Specific Antigen (PSA). PMs may be purified from lung primary tissue or lung cells (such as A549 cells) using the monoclonal antibodies anti-CD-138 and anti-CD-151. Membranes may also be purified from pancreatic primary tissue and pancreatic cells (such as BxPC-3 cells) by using the monoclonal antibody anti-CD-138 which recognizes syndecan-1. In another example, membranes from melanomatous skin tissue may be purified using the monoclonal antibody 9.2.27, which recognizes chondroitin sulfate-like proteoglycan.

The selected antibodies might also bind PM antigens located in a subdomain of the PM, which includes the apical and basolateral PM subdomains. Such antibodies could be used to select for a particular subdomain, or combined to isolate a more representative PM. This is particularly important in the case of epithelial tissues as they are polarized cells, implying that their PMs are not homogenous but rather separated into two distinct domains, the apical and baso-lateral domains. Therefore, to successfully purify an intact PM and not just a portion of it, the selected antibodies should be used for purification of both subdomains. In the embodiment described herein, high-specificity antibodies meeting these criteria and allowing the purification of the two subdomains of an epithelial PM when used in combination were the anti-CEA and the anti-ESA antibodies.

In another embodiment, since the expression of the ESA antigen is not restricted to colon tissue but rather detected in all epithelial tissues, the anti-ESA antibody is useful for purifying PMs of any kind of human epithelial cell and/or tissue. In yet another embodiment, as the expression of the CEA antigen is likewise not restricted to colonic tumor cells, but rather detected in many primary human tumors (such as pancreatic tumors) and cell lines (such as pancreatic adenocarcinoma cell line BxPC-3), the anti-CEA antibody is useful for purifying PMs of any cell type and/or tumor tissue where the CEA antigen is expressed.

Antibodies useful in the practice of the invention are typically directed against PM antigenic determinants found in normal cells or abnormal cells (for example, malignant cells or non-malignant cells) or both. Malignant cells include, but are not limited to, cells derived from, breast, ovarian, and lung carcinoma, melanoma, sarcoma, glioblastoma, and cancers of the gastrointestinal tract and the reticuloendothelial system. Cells associated with non-malignant diseases include, but are not limited to those associated with cardiovascular, neurological, pulmonary, autoimmune, gastrointestial, metabolic, and other disorders. Normal cells include, but are not limited to, matched cell type populations for comparison to abnormal cells. Cells may be of essentially any cell type, and of homogenous or non-homogenous populations or preparations. Cell populations, such as of malignant cells, may be obtained from the tissue found in bone marrow, peripheral blood, from pleural and peritoneal effusions and other body fluid compartments, such as urine, cerebrospinal fluid, lymph or from solid tumors in tissues and/or organs, for example liver, lymph nodes, spleen, lung, pancreas, bone tissue, the central nervous system, organs or tissues of the digestive system, prostate, skin, and/or mucous membranes.

Preferred sources of materials used as biological samples for the methods of the invention include any primary tissue, cell line, or constituent cell thereof. In one embodiment described in the examples herein, PMs were purified from primary human colon tumor tissue and a corresponding selected cultured cell model, CaCo-2, which was derived from a human adenocarcinoma colon tumor. Immunopurification of PM from primary tissues can be facilitated by the knowledge of the optimum conditions established for the immunoisolation of the PMs of the cultured cell model. In either case, it is preferable that the cells of the biological sample be prepared as a monodisperse, or single cell suspension, as in Examples 1 and 3, or through the use of any other art-known methods for producing an appropriate single cell suspension.

The methods further provide for the antibody/antigen complexes to be recovered from the biological sample, thereby purifying PM bound in or interacting with the antibody/antigen complex. Recovery may be accomplished by any method known in the art, but should be selective for the antibody/antigen complex. In preferred embodiments, the antibodies are also bound to secondary antibodies, and/or to an insoluble affinity support reagent such as magnetic beads or sepharose beads. While other supports, such as cellulose or acrylamide have been used for organelle immunoisolation (Devaney and Howell (1985) EMBO J. 4: 3123-3130 (PMID: 4092679)), such supports are less preferable as they are incompatible with protein identification methods such as mass spectrometry analysis because of non-specific protein binding. Such non-specific binding of proteins masks and prevents the identification of the proteins of interest. Indeed, the disclosed purification methods enable mass spectrometry analysis and protein identification directly on the purified PM fraction, as magnetic and sepharose supports exhibit low non-specific protein binding.

Paramagnetic beads are a preferred embodiment for use as an insoluble affinity support reagent. Paramagnetic beads are preferable to sepharose beads, but paramagnetic microbeads are most preferable. Beads and microbeads may be of any shape, but preferably are roughly spherical. The approximate diameter or longest axis of the reagent is preferably about 5 μm or less, or 4.5 μm or less, more preferably it is about 4.4 μm or less, 4.3 μm or less, 4.2 μm or less, 4.1 μm or less, 4.0 μm or less, 3.9 μm or less, 3.8 μm or less, 3.7 μm or less, 3.6 μm or less, 3.5 μm or less, 3.4 μm or less, 3.3 μm or less, 3.2 μm or less, 3.1 μm or less, 3.0 μm or less, or 2.9 μm or less. Even more preferably it is 2.8 μm or less, 2.7 μm or less, 2.6 μm or less, 2.5 μm or less, 2.4 μm or less, 2.3 μm or less, 2.2 μm or less, 2.1 μm or less, 2.0 μm or less, 1.9 μm or less, 1.8 μm or less, 1.7 μm or less, 1.6 μm or less, 1.5 m or less, 1.4 μm or less, 1.3 μm or less, 1.2 μm or less, 1.1 μm or less, 1.0 μm or less, 900 nm or less, 800 nm or less, 700 nm or less, 600 nm or less, 500 nm or less, 400 nm or less, 300 nm or less, or 200 nm or less. Most preferably it is 100 nm or less, 90 nm or less, 80 nm or less, 70 nm or less, 60 nm or less, 50 nm or less, 40 nm or less, 30 nm or less, 20 nm or less, 10 nm or less, or 5 nm or less.

The PM fraction isolated should be highly pure, as measured by standard biochemical or cell biological methods (such as electron microscopy). In preferred embodiments the PM fraction is at least 70% pure. More preferably the PM fraction is at least 75%, 80%, or 85% pure. More preferable still the PM fraction is at least 86%, 87%, 88%, 89%, 90%, 91%, 92%, 93%, 94%, or 95% pure. Most preferable this fraction is of a purity of at least 96%, 97%, 98%, 99%, or better.

In an alternative embodiment, the method provides for selection of cells of the biological sample through FACS using a PM antigen-binding antibody or antigen-binding fragment thereof at least prior to the recovering of the antigen/antibody complexes, if not prior to contacting the biological sample with the PM antigen-binding antibody or antigen-binding fragment thereof to be used for PM immunoisolation. Exemplary antibodies for use in FACS are anti-CD-104, anti-CD-138, and anti-CD-164.

In one working example, a monoclonal antibody (or antigen-binding fragment thereof) specific to a PM cell surface antigen is incubated with a suspension of dissociated cells obtained from a cell culture or primary tissue. If desired, a second monoclonal antibody directed to a second, distinct cell surface antigen might be also be employed. After washing unbound antibodies, the cells are lysed under mild conditions and subjected to differential centrifugation to remove unbroken cells and large aggregates. The resulting post-nuclear supernatant containing the PMs is then incubated with magnetic beads previously coupled to secondary antibodies that recognize the antibodies previously bound to the PM. Suitable magnetic beads are commercially available from Dynal (Oslo, Norway). Magnetic bead/PM immunocomplexes are then separated from other organelles using standard magnetic separation methods. When desired, the isolated PM can be separated from the magnetic beads using standard methods known in the art. Isolated PMs are subsequently characterized and assayed according to standard biochemical and morphological methods. Such biochemical methods include SDS-PAGE and Western blotting (Lanoix et al. (1999) EMBO J 18: 4935-4948 (PMID: 10487746)). Exemplary morphological methods for characterizing purified PMs include using standard electron microscopy methodologies (Lanoix et al. (1999) supra); Lanoix et al. (2001) Journal of Cell Biology 155: 1199-1212 (PMID: 11748249)). For example, for morphological quantitation, electron micrographs are overlaid with a lattice and the relative proportions of PMs and contaminating organelles are determined by the standard point-counting method described by Weibel ((1969) Intern. Rev. Cytol.26: 235-302 (PMID: 4899604)).

Purified PMs prepared as described above are suitable for direct mass spectrometry analysis following electrophoresis, such as 2-dimensional gel electrophoresis (see Example 6), and may subsequently be used for protein identification.

### EXAMPLES

The features and other details of the invention will now be more particularly described and pointed out in the following examples describing preferred techniques and experimental results. These examples are provided for the purpose of illustrating the invention and should not be construed as limiting.

### Example 1

### Preparation of a Single Cell Suspension from Cultured CaCo-2 Cells

CaCo-2 cells were cultured according to ATCC product information data sheet for the cell line HTB-37. Confluent cells were washed using 20 ml of D-PBS (Multicell) and then incubated in 10 ml of cell dissociation solution (Sigma C-5914) for 10 minutes at 37° C. 20 ml of Eagle-MEM growth media (supplemented with 16% FBS and penicillin/streptomycin) was added and the cells were dissociated by pipetting. After centrifugation at 280×g for 5 minutes at 4° C., the cells were washed twice in a volume of 25 ml Eagle-MEM growth medium. The number of viable cells was determined by trypan blue stain exclusion, and 85% of the cells were found to be viable. The cells were then resuspended in 1 ml ice cold Eagle-MEM (without FCS or penicillin/streptomycin), the cell suspensions were pooled (9 ml, 125 million of cells), the volume was split into two equal parts (2×4.5 ml), and then equally divided into 15 ml Falcon tubes.

### Example 2

### Immunomagnetic Purification of Plasma Membrane from Cultured Cells

To 4.5 ml of a single cell suspension of CaCo-2 cells (approximately 62.5 million cells) (see Example 1), 120 μg (600 μl) of ESA Ab-3 monoclonal antibody (anti-ESA) (200 μg/ml) (NeoMarkers) and 120 μg (600 μl) of anti-CEA antibody (200 μg/ml) was added. The cells were incubated for 30 minutes at 4° C. in a Dynal Sample Mixer (Dynal Biotech, Lake Success, N.Y.) set to 15 rpm, and the cells were then collected by centrifugation at 280×g for 5 minutes at 4° C. Each cell pellet was resuspended in 5 ml of PBS blocking buffer, PVP40T (2 mg/ml, Sigma, St.Louis, Mo.), non-fat dry milk (0.5 mg/ml), and antiproteases (one tablet of protease inhibitor cocktail from Boehringer Mannheim/50 ml) and the cells were collected again by centrifugation at 280×g for 5 minutes at 4° C. The cell pellets were resuspended in 5 ml of PBS blocking buffer and lysed on ice by vigorous aspiration with a 1 cc syringe mounted with a 26-gauge needle. Cell lysis was monitored by trypan blue exclusion. The lysates were then pooled and centrifuged at 900×g for 20 minutes at 4° C. Post-nuclear supernatants (PNS) were collected and the amount of unbroken cells and nuclei in the PNS were again monitored by trypan blue exclusion. When nuclei or unbroken cells were no longer detected in the PNSs, 400 μl of Dynabeads Pan Mouse IgG (Dynal Biotech, Lake Success, N.Y.) were added. After incubation for 60 minutes at 4° C. in a Dynal Sample Mixer (Dynal Biotech, Lake Success, N.Y.) set to 15 rpm, the beads were washed twice with 7 ml of PBS blocking buffer. After each wash, the beads were resuspended by vortexing. For the final wash, the beads were resuspended in 7 ml PBS. The supernatant was next split into equal volumes and the beads were collected using a magnet. Standard biochemical and morphological analyses were conducted to assess the purity of the PM fraction.

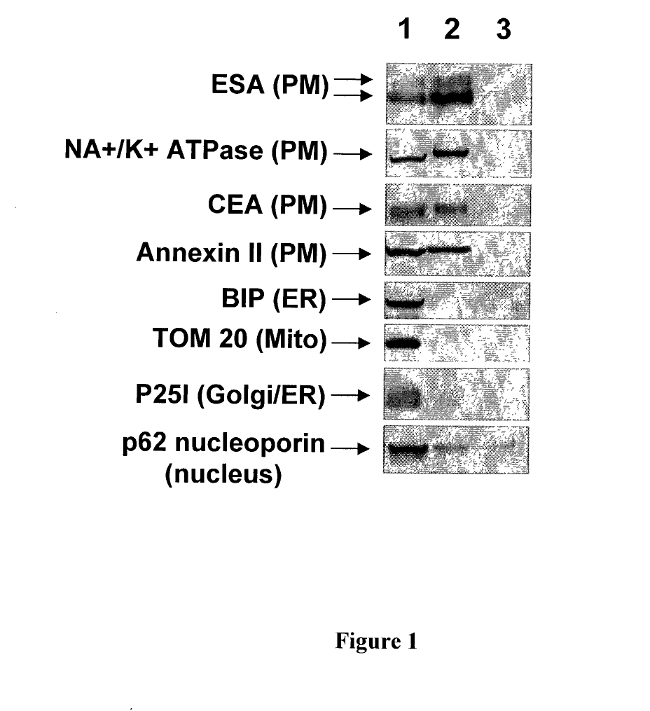

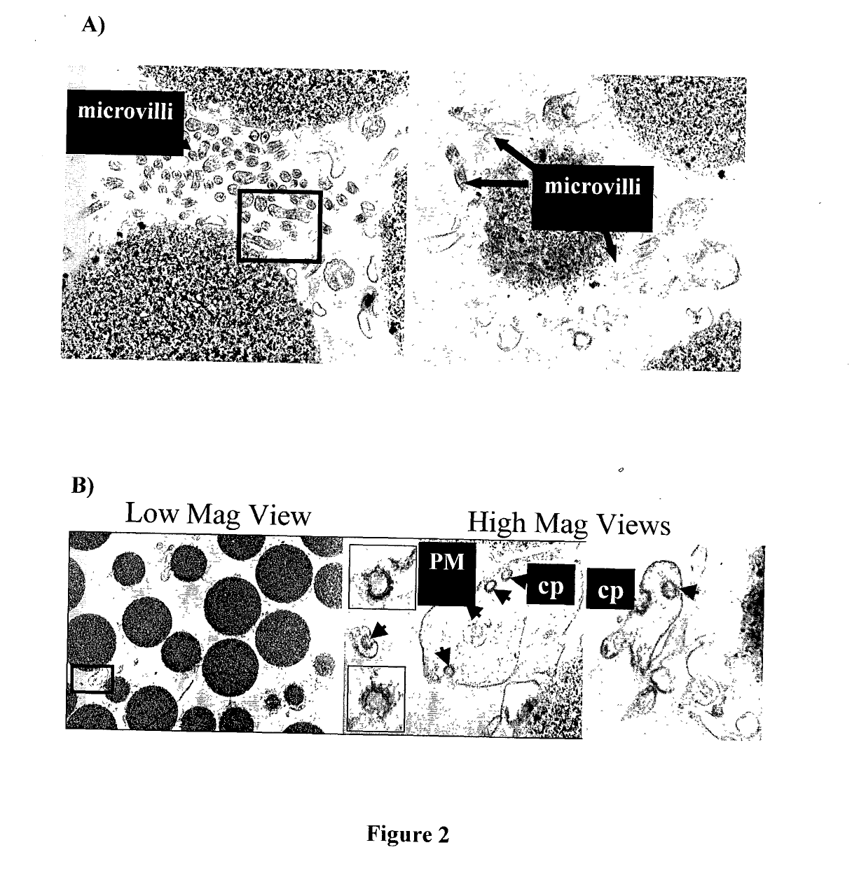

As shown in FIG. 1, standard western blotting of the material bound to the magnetic beads showed that the anti-ESA/CEA bead fraction (see lane 2) contained PMs as assessed by the detection of four known PM markers: ESA, Na⁺/K⁺ ATPase, CEA, and annexin II. The presence of antigens localized in different sub-domains of the PM (the CEA (apical domain) and ESA and Na⁺/K⁺ ATPase (basolateral domain) proteins) indicated that the described immunoisolation procedure permitted the purification of the entire PM, with its integrity intact. In addition, none of the contaminating organelle markers were readily detected in the ESA/CEA fraction, indicating that this PM fraction is indeed extremely pure. The morphological analysis-of the ESA/CEA bead fraction by electron microscopy (FIG. 2) fully supported the above-described biochemical results, since microvilli and clathrin-coated structures were identified in the material bound to the beads. Both microvilli and clathrin-coated structures are well-known morphological hallmarks of the PMs. Indeed, other structures representing contaminating organelles were not observed in the bead fraction.

### Example 3

### Method for Cell Suspension from Primary Colonic Tissue

A cell suspension was prepared by dissolving a primary colon tumor mass in an enzymatic solution that included collagenase and elastase (Worthington Tissue Dissociation Guide, Worthington Biochemical Corp., Freehold, N.J., 1990) in order to obtain dissociated cells. Frozen human colon cancer tissue biopsy (2 cubes of approximately 150 mg each, Ardais Corporation) was thawed at room temperature for 30 minutes. The cube was then washed with 10 ml of 25 mM Hepes pH 7.4/D-MEM medium/10% FBS, and 5 ml of a collagenase (300 U/ml) and elastase (10 U/ml) solution containing trypsin inhibitors was injected into the cube using a 1 ml syringe mounted with a 26 gauge needle. Five ml of this solution was then added and the suspension was stirred vigorously for 20 minutes at 37° C. The incubation medium was centrifuged at 300×g for 5 minutes and the supernatant containing the dissociated cells was collected. The remaining tumor mass was then incubated with 5 ml of fresh dissociation solution for 20 minutes at 37° C., and the supernatant containing the dissociated cells was collected. The remaining tumor mass was similarly treated with 5 ml of fresh dissociation solution for 20 minutes at 37° C. Cell dissociation was monitored by phase contrast microscopy. The supernatants were pooled, the sample was then washed twice with E-MEM medium by centrifugation at 300×g for 5 minutes and the pellet was resuspended in 1 ml of E-MEM medium.

### Example 4

### Immunomagnetic Plasma Membrane Purification from Primary Tissue Using Paramagnetic Beads

To a tube containing 9 ml of dissociated cells (360 million cells, Example 3), 200 μg (100 μl) of ESA-Ab-3 monoclonal antibody (anti-ESA) (200 μg/ml) (NeoMarkers) and 200 μg (100 μl) of an anti-CEA antibody was added. The sample was then incubated for 30 minutes at 4° C. in a Dynal Sample Mixer (Dynal Biotech, Lake Success, N.Y.) set to 15 rpm. After centrifugation at 280×g for 5 minutes at 4° C., the pellet was washed twice in a volume of 10 ml of PBS blocking buffer. The cells were resuspended in 25 ml of PBS blocking buffer and then transferred to a Parr bomb (Parr cell disruption bomb: Parr Instrument Company, model number 4639). The cells were disrupted using the Parr bomb at 350 psi for 15 minutes on ice, and a 100 μL aliquot examined by light microscopy with trypan blue staining to evaluate efficiency of homogenization. Cell lysates were then centrifuged at 900×g for 5 minutes at 4° C., and the PNS was collected. The amount of unbroken cells and nuclei in the PNS was monitored by trypan blue exclusion. To the PNS, 500 μl of Dynabeads Pan Mouse IgG (Dynal Biotech, Lake Success, N.Y.) was added. The mixture was incubated for 60 minutes at 4° C. in a Dynal Sample Mixer (Dynal Biotech, Lake Success, N.Y.) set to 15 rpm. The beads were then washed two times with 10 ml of PBS blocking buffer using a magnet. After each wash, the beads were resuspended by vortexing the mixtures 5 seconds each time. For the final wash, the beads were resuspended in 10 ml PBS. The beads were then collected using a magnet, snap-frozen in liquid nitrogen and stored at −80° C. The purity of the PM fraction was assessed as described above.

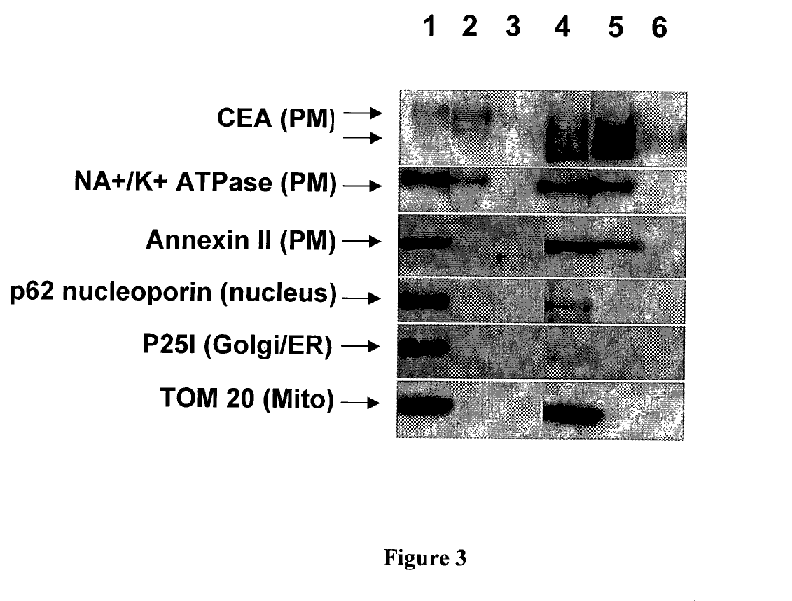

When employing optimum conditions previously determined for the CaCo-2 cell line, the resulting PM fraction (FIG. 3, lane 2) was shown to contain three known PM markers Na⁺/K⁺ ATPase, CEA, and annexin II and minimal contamination by other organelles indicating an extremely high level of purity. Since CEA and Na⁺/K⁺ ATPase proteins were both detected in the PM fraction, this result indicated that, like the PM fraction purified from the cultured CaCo-2 cells, the entire PM was also successfully isolated.

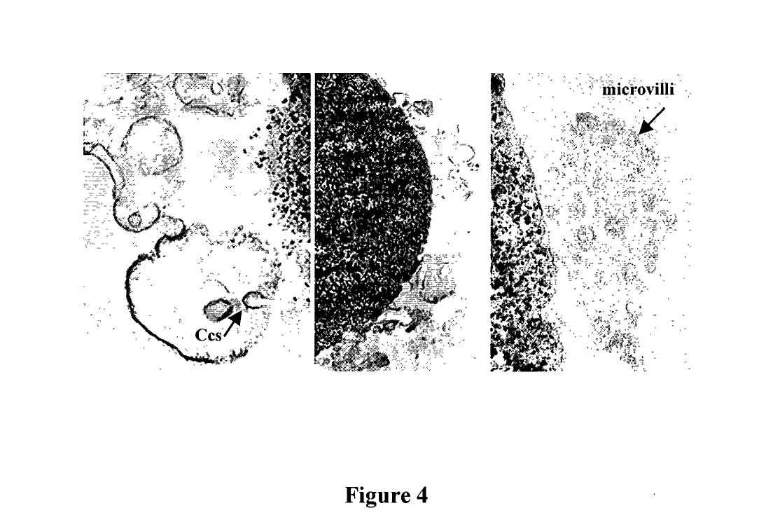

The morphological characterization of the ESA/CEA bead fraction (FIG. 4) confirmed the biochemical analysis, since the electron microscopy showed that the material bound to the beads revealed the presence of microvilli as well as clathrin-coated structures. Other contaminating organelle structures were not observed in this PM fraction.

### Example 5

### Immunomagnetic Isolation of Plasma Membrane From Cell Culture or Fresh Tissue Using Paramagnetic Microbeads

Solutions were prepared as follows. PBS/PVP: 2 mg/mL PVP-40T (Sigma, St, Louis, Mo.) was added to 500 mL D-PBS, and filtered under vacuum. For PBS/PVP/milk, 0.5 mg/mL non-fat dry milk (Instant skim milk powder: Nestle, Product Number 6500011132.3) was added to PBS/PVP and filtered under vacuum. Sucrose-tris (ST) buffer: 250 mM sucrose/10 mM tris, pH 7.4, and filtered under vacuum. For ST/antiprotease, 1 tablet antiprotease (Complete protease inhibitor cocktail tablets: Roche, Catalog Number 1836145) was added to 50 mL ST buffer. For PBS/PVP/antiprotease, 1 tablet antiprotease (Complete protease inhibitor cocktail tablets: Roche, Catalog Number 1836145) was added to 50 mL PBS/PVP and degassed by stirring under vacuum for 15 minutes immediately before use. For PBS/PVP/milk/antiprotease, 1 tablet antiprotease (Complete protease inhibitor cocktail tablets: Roche, Catalog Number 1836145) was added to 50 mL PBS/PVP/milk and degassed as above. In addition, a 33% (1.28 M) sucrose solution (Fisher, Catalog Number S5-500) was prepared.

A cell suspension was prepared for immunoisolation. Fifty million cells were placed in a 50 mL tube. The volume was brought up to 40 ml with PBS/PVP/milk, and then spun at 1200 rpm (300 g) for 5 minutes at 4° C. After removing the supernatant, the cells were resuspended in 9.25 mL PBS/PVP/milk containing 750 μL primary antibody (1 μg anti-ESA (ESA Ab-3, IgG₁, 200 μg/mL; Neomarkers, Fremont, Calif., Catalog Number MS-181-P)/million cells; plus 2 μg anti-CEA (CEA Ab-3, IgG₂ₐ, 200 μg/mL; Neomarkers, Fremont, Calif., Catalog Number MS-613-P)/million cells), bringing the total volume up to 10 mL (5 million cells/mL).

The cells were incubated for 30 minutes at 4° C. and 15 rpm in Dynal Sample Mixer (Dynal Biotech, RKDYNAL 10111). Then, PBS/PVP/milk was added, bringing the volume up to 50 mL, and the cells were centrifuged at 1200 rpm (300 g) for 5 minutes at 4° C. The supernatant was removed and the pellet gently resuspended in 10 mL PBS/PVP/milk. After incubation, PBS/PVP/milk was again added to bring the volume to 50 mL, and the cells were centrifuged and gently resuspended a second time in 9.375 mL PBS/PVP/milk.

50 μL microbeads (MACS Goat anti-mouse IgG MicroBeads (Miltenyi Biotec, Auburn, Calif., Catalog Number 48-401))/million cells=625 μL beads were added to the cell suspension, bringing the total volume to 10 mL, and incubated for 30 minutes at 4° C. and 15 rpm in a Dynal Sample Mixer. D-PBS was added until the total volume reached 50 mL, and the cells were centrifuged at 1200 rpm (300 g) for 5 minutes at 4° C. The pellet was gently resuspended in 10 mL D-PBS, and a 50 μL aliquot taken. Using the aliquot, a cell count was performed and the cell viability checked by trypan blue exclusion. D-PBS was again added to bring the suspension to a total volume of 50 mL, and cells likewise centrifuged a second time as above, and the pellet was gently resuspended in 5 mL ST/antiprotease.

The sample was then snap frozen in liquid nitrogen, and stored at −80° C. overnight. The sample was thawed by placement in a beaker of water at room temperature accompanied by stirring. The thawed sample was transferred to a Parr bomb (Parr cell disruption bomb: Parr Instrument Company, model number 4639). The tube that contained the sample was washed twice with 2.5 mL ST/antiprotease, and the washes were also transferred to the Parr bomb (total volume=10 mL): 5 million cells/mL. The cells were disrupted using the Parr bomb at 350 psi for 15 minutes on ice, and a 100 μL aliquot examined by light microscopy with trypan blue staining to evaluate efficiency of homogenization.

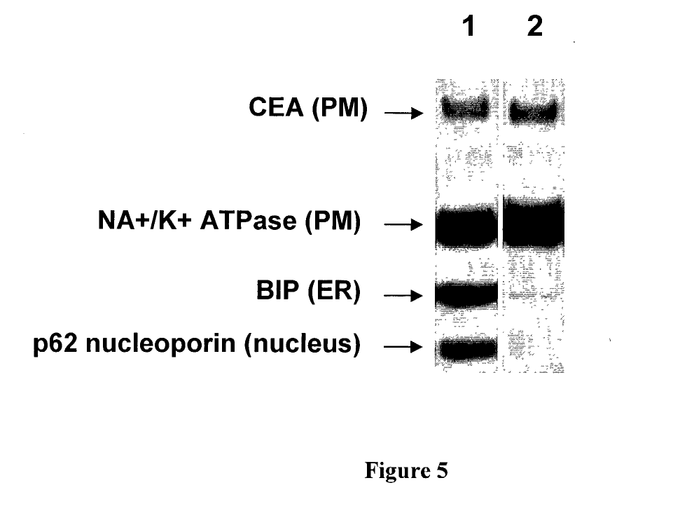

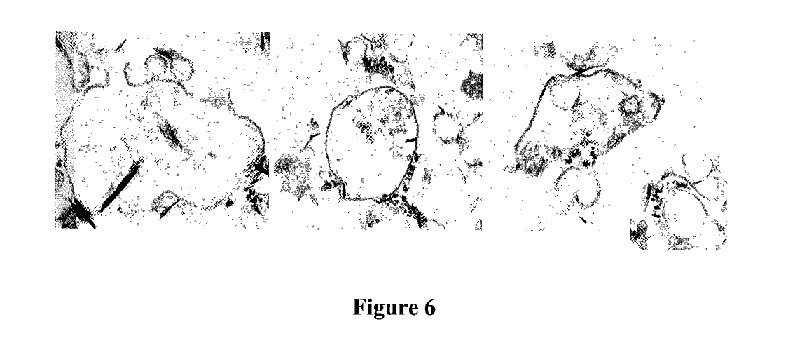

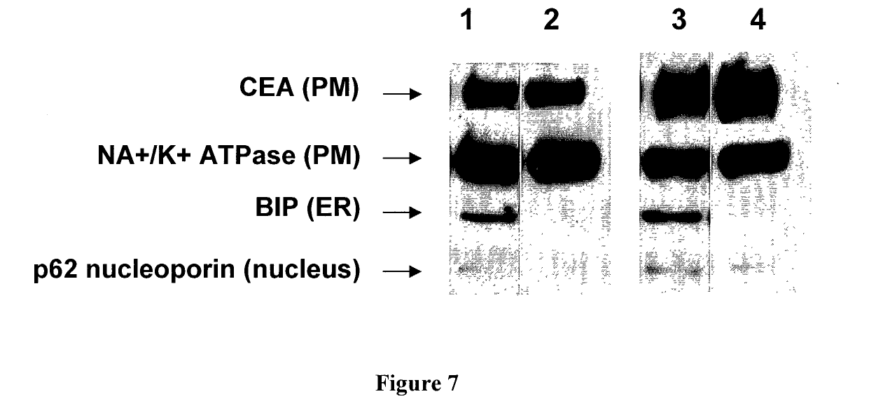

In an environmental cold room (4° C.), an LS separation column (Miltenyi Biotec, Catalog Number 42-401) was placed on a MidiMACS separation unit (magnet) (Miltenyi Biotec, Catalog Number 42-302) and the column was equilibrated with 3 mL PBS/PVP/milk/antiprotease, allowing the flow to stop between additions of buffer or sample to column (it is designed as a stop-flow column and will not dry out). The sample was applied to the column, and the effluent collected as a negative (non-magnetic) fraction or “flow-through” (100 μL flow-through was kept for analysis). The column was then washed, filling the column chamber as full as possible to rinse material from sides, with 3×10 mL PBS/PVP/antiprotease, and the effluent collected as wash. The column was removed from the magnet and the positive (magnetic) fraction eluted with 3.5 mL PBS/PVP/antiprotease into a 5 mL tube. The eluant was then transferred to a SW 60 Ti ultracentrifuge tube (Optima Ultracentrifuge: Beckman Coulter, Model XL-100K; Ultracentrifuge rotor type SW 60 Ti: Beckman Coulter; Ultra-clear centrifuge tubes (4 mL): Beckman, Catalog Number 344062), and a cushion of 50 μL 33% (1.28 M) sucrose placed at the bottom of the tube. The sample was centrifuged at 50,000 rpm for 30 minutes at 4° C. to pellet the membranes, and the supernatant removed (including the sucrose cushion since the membranes are pelleted at the bottom of the tube). The pellet was resuspended in 100 μL ST/antiprotease with a micropipettor and gently vortexing and prepared for SDS-PAGE or snap-freeze and storage at −80° C. The morphology and biochemical characterization of the purified PMs is shown in FIGS. 5, 6, and 7.

### Example 6

### Analysis of Plasma Membrane Components by Mass Spectrometry

Plasma membrane proteins (for example, from Example 5) are first dissolved in a lysis/rehydration buffer, centrifuged, and then applied to an IPG strip (IPGphor Amersham Pharmacia) and separated in the first dimension, according to the manufacturer's specifications. Antibodies attached to the PM antigens are separated from the beads, as the beads are too large to migrate through the gel. The strip is then removed and equilibrated and the sample is run in the second dimension on SDS-PAGE. After separation in the second dimension, the gel is stained with a silver stain and scanned. The spots are then excised and placed in a 96 well plate. The gel pieces are then placed on a Massprep (liquid handler; Micromass UK) and are desalted and digested with trypsin according to the manufacturer's instructions. Excess liquid is then removed from the gel piece, and deposited into A) wells of a 96 well plate and B) wells of a 96 well target plate for analysis by MALDI-TOF mass spectrometry (Micromass UK).

All publications and patents mentioned in this specification are herein incorporated by reference to the same extent as if each individual publication or patent was specifically and individually indicated to be incorporated by reference.

Other Embodiments

From the foregoing description, it will be apparent that variations and modifications may be made to the invention described herein to adopt it to various usages and conditions. Such embodiments are also within the scope of the following claims.

What is claimed is:

1. A method for purifying a plasma membrane, comprising:
(a) contacting a biological sample with one or more antibodies or antigen-binding fragments thereof that specifically bind to a plasma membrane antigen, thereby forming an antibody/plasma membrane complex; and
(b) recovering said antibody/plasma membrane complex, thereby purifying said plasma membrane from the biological sample.

2. The method of claim 1, wherein step (a) further comprises incubating said antibody/plasma membrane complex with an insoluble affinity support reagent that specifically binds said antibody or antigen-binding fragment thereof.

3. The method of claim 1, wherein the plasma membrane is a cell surface plasma membrane.

4. The method of claim 1, wherein said antibody is a polyclonal antibody.

5. The method of claim 1, wherein said antibody is a purified polyclonal antibody.

6. The method of claim 1, wherein said antibody is an affinity purified polyclonal antibody.

7. The method of claim 1, wherein said antibody is a monoclonal antibody.

8. The method of claim 1, wherein said antibody is directed to a subdomain antigen of the plasma membrane.

9. The method of claim 8, wherein said antibody is directed to an apical or a basolateral plasma membrane domain antigen.

10. The method of claim 1, wherein said antibody comprises anti-ESA or anti-CEA.

11. The method of claim 1, wherein said antibody comprises at least anti-ESA and anti-CEA.

12. The method of claim 11, wherein, prior to the recovering of claim 1, the biological sample has undergone fluorescence activated cell sorting utilizing one or more of the following: anti-CD-164, anti-CD-138, or anti-CD-104.

13. The method of claim 1, wherein said biological sample is a cell culture.

14. The method of claim 1, wherein said biological sample is a primary tissue.

15. The method of claim 1, wherein said biological sample is a population of tumor cells.

16. The method of claim 15, wherein said tumor cells are carcinoma cells.

17. The method of claim 16, wherein said carcinoma cells are from a carcinoma having a primary site is breast, ovary, stomach, intestine, colon, brain, or lung tissue.

18. The method of claim 13, 14, 15, 16, or 17, wherein the biological sample is prepared as a single cell suspension prior to the contacting of claim 1.

19. The method of claim 2, wherein said insoluble affinity support is a paramagnetic bead.

20. The method of claim 19 wherein said paramagnetic bead is of about 4.5 μm diameter or less.

21. The method of claim 19 wherein said paramagnetic bead is of about 3.0 μm diameter or less.

22. The method of claim 19 wherein said paramagnetic bead is of about 2.8 μm diameter or less.

23. The method of claim 19 wherein said paramagnetic bead is of about 2.6 μm diameter or less.

24. The method of claim 19 wherein said paramagnetic bead is of about 2 μm diameter or less.

25. The method of claim 19 wherein said paramagnetic bead is of about 1 μm diameter or less.

26. The method of claim 19 wherein said paramagnetic bead is of about 500 nm diameter or less.

27. The method of claim 19 wherein said paramagnetic bead is of about 100 nm diameter or less.

28. The method of claim 19 wherein said paramagnetic bead is of about 50 nm diameter or less.

29. The method of claim 2, wherein said insoluble affinity support is a sepharose bead.

30. A method for analyzing plasma membrane polypeptides by mass spectrometry, the method comprising the steps of:
(a) purifying said plasma membrane according to the method of claim 1; and
(b) analyzing purified plasma membrane polypeptides by mass spectrometry.

31. A purified plasma membrane fraction.

32. The purified plasma membrane fraction of claim 31, wherein said fraction is 95% pure or greater.

33. The purified plasma membrane fraction of claim 31, wherein said fraction is 90% pure or greater.

34. The purified plasma membrane fraction of claim 31, wherein said fraction is 85% pure or greater.

35. The purified plasma membrane fraction of claim 31, wherein said plasma membrane fraction is obtained from membranes of a cell culture.

36. The purified plasma membrane fraction of claim 31, wherein said plasma membrane fraction is obtained from plasma membranes of a primary tissue.

37. The purified plasma membrane fraction of claim 31, wherein said plasma membrane fraction is obtained from plasma membranes of a population of tumor cells.

38. The purified plasma membrane fraction of claim 31, wherein said fraction includes plasma membranes having microvilli.

39. The purified plasma membrane fraction of claim 31, wherein said fraction includes plasma membranes having a clathrin-coated morphology.

40. A purified plasma membrane/magnetic bead immunocomplex.

41. The purified plasma membrane/magnetic bead immunocomplex of claim 40, wherein said immunocomplex further includes one or more antibodies.

42. The purified plasma membrane/magnetic bead immunocomplex of claim 40, wherein said plasma membrane is obtained from membranes of a cell culture.

43. The purified plasma membrane/magnetic bead immunocomplex of claim 40, wherein said plasma membrane fraction is obtained from plasma membranes of primary tissue.

44. The purified plasma membrane/magnetic bead immunocomplex of claim 40, wherein said plasma membrane fraction is obtained from plasma membranes of a population of tumor cells.

45. The purified plasma membrane/magnetic bead immunocomplex of claim 40, wherein said plasma membrane fraction includes plasma membranes having microvilli.

46. The purified plasma membrane/magnetic bead immunocomplex of claim 40, wherein said plasma membrane fraction includes plasma membranes having a clathrin coated morphology.

47. The purified plasma membrane/magnetic bead immunocomplex of claim 40, wherein said magnetic bead is a paramagnetic bead.

48. The purified plasma membrane/magnetic bead immunocomplex of claim 40, wherein said magnetic bead is a paramagnetic microbead.
